(19) United States
(12) Patent Application Publication
(10) Pub. No.: US 2009/0206275 A1
(43) Pub. Date: Aug. 20, 2009

(54) ACCELERATOR PARTICLE BEAM APPARATUS AND METHOD FOR LOW CONTAMINATE PROCESSING

(75) Inventors: Francois J. Henley, Aptos, CA (US); Albert Lamm, Suisun City, CA (US); Adam Brailove, San Jose, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Silcon Genesis Corporation, San Jose, CA (US)

(21) Appl. No.: 12/244,687

(22) Filed: Oct. 2, 2008

Related U.S. Application Data
(60) Provisional application No. 60/997,684, filed on Oct. 3, 2007.

(51) Int. Cl. H01J 27/00; A61N 5/00

(52) U.S. Cl. 250/424; 250/423 R; 250/492.1

(57) ABSTRACT

A system of introducing a particle beam such as a linear accelerator particle beam for low contaminate processing. The system includes an accelerator apparatus configured to generate a first particle beam including at least a first ionic specie in an energy level of 1 MeV to 5 MeV or greater. Additionally, the system includes a beam filter coupled to the linear accelerator apparatus to receive the first particle beam. The beam filter is in a first chamber and configured to generate a second particle beam with substantially the first ionic specie only. The first chamber is associated with a first pressure. The system further includes an end-station including a second chamber coupled to the first chamber for extracting the second particle beam. The second particle beam is irradiated onto a planar surface of a bulk workpiece loaded in the second chamber for implanting the first ionic specie. The second chamber is associated with a second pressure that is higher than the first pressure. Optional beam scanning can also be added between the beam filter and the end-station.

### CROSS-REFERENCE TO RELATED APPLICATION

The instant nonprovisional patent application claims priority to U.S. Provisional Patent Application 60/997,684 filed Oct. 3, 2007, which is incorporated by reference in its entirety herein for all purposes.

### BACKGROUND OF THE INVENTION

The present invention relates generally to technique including a method and a structure for forming substrates using a layer transfer technique. More particularly, the present invention provides a method and system using a linear accelerator particle beam or a particle beam from another type of accelerator such as a cyclotron or the like, with low contaminate process for the manufacture of thick free-standing semiconductor films for a variety of applications including photovoltaic cells. But it will be recognized that the invention has a wider range of applicability; it can also be applied to other types of applications such as for three-dimensional packaging of integrated semiconductor devices, photonic or optoelectronic devices, piezoelectronic devices, flat panel displays, microelectromechanical systems (“MEMS”), nano-technology structures, sensors, actuators, integrated circuits, biological and biomedical devices, and the like.

From the beginning of time, human beings have relied upon the “sun” to derive almost all useful forms of energy. Such energy comes from petroleum, radiant, wood, and various forms of thermal energy. As merely an example, human being have relied heavily upon petroleum sources such as coal and gas for much of their needs. Unfortunately, such petroleum sources have become depleted and have lead to other problems. As a replacement, in part, solar energy has been proposed to reduce our reliance on petroleum sources. As merely an example, solar energy can be derived from “solar cells” commonly made of silicon.

The silicon solar cell generates electrical power when exposed to solar radiation from the sun. The radiation interacts with atoms of the silicon and forms electrons and holes that migrate to p-doped and n-doped regions in the silicon body and create voltage differentials and an electric current between the doped regions. Depending upon the application, solar cells have been integrated with concentrating elements to improve efficiency. As an example, solar radiation accumulates and focuses using concentrating elements that direct such radiation to one or more portions of active photovoltaic materials. Although effective, these solar cells still have many limitations.

As merely an example, solar cells rely upon starting materials such as silicon. Such silicon is often made using either polysilicon (i.e. polycrystalline silicon) and/or single crystal silicon materials. These materials are often difficult to manufacture. Polysilicon cells are often formed by manufacturing polysilicon plates. Although these plates may be formed effectively, they do not possess optimum properties for highly effective solar cells. Single crystal silicon has suitable properties for high grade solar cells. Such single crystal silicon is, however, expensive and is also difficult to use for solar applications in an efficient and cost effective manner. Additionally, both polysilicon and single-crystal silicon materials suffer from material losses during conventional manufacturing called “kerf loss”, where the sawing process eliminates as much as 40% and even up to 60% of the starting material from a cast or grown boule and singulate the material into a wafer form factor. This is a highly inefficient method of preparing thin polysilicon or single-crystal silicon plates for solar cell use.

Generally, thin-film solar cells are less expensive by using less silicon material but their amorphous or polycrystalline structure are less efficient than the more expensive bulk silicon cells made from single-crystal silicon substrates. These and other limitations can be found throughout the present specification and more particularly below.

From the above, it is seen that techniques for forming suitable substrate materials of high quality and low cost are highly desired.

### BRIEF SUMMARY OF THE INVENTION

The present invention relates generally to technique including a method and a structure for forming substrates using a layer transfer technique. More particularly, the present invention provides a method and system using a linear accelerator particle beam or a particle beam from another type of particle accelerator such as a cyclotron of the like, with low contaminate process for the manufacture of thick free-standing semiconductor films for a variety of applications including photovoltaic cells. But it will be recognized that the invention has a wider range of applicability; it can also be applied to other types of applications such as for three-dimensional packaging of integrated semiconductor devices, photonic or optoelectronic devices, piezoelectronic devices, flat panel displays, microelectromechanical systems (“MEMS”), nano-technology structures, sensors, actuators, integrated circuits, biological and biomedical devices, and the like.

In a specific embodiment, the present invention provides a system of introducing a particle beam for low contaminate processing. The system includes a particle accelerator with at least an ion source for generating a plurality of charged particles and an apparatus for accelerating and confining the plurality of charged particles in a first particle beam. The first particle beam includes at least a first ion specie and is outputted to a first exit aperture in a first spatial direction. Additionally, the system includes a beam filter apparatus coupled to the first exit aperture to receive the first particle beam. The beam filter apparatus includes a first chamber of a first mass-selection device to process the first particle beam and generate a second particle beam. The second particle beam includes substantially the first ionic specie and being outputted to a second exit aperture in a second spatial direction different from the first spatial direction. Moreover, the system includes an end-station with a second chamber coupled to the beam filter apparatus to receive the second particle beam. The second chamber is configured to house a workpiece having a planar surface configured to receive the second particle beam for implanting the first ionic specie.

In an alternative specific embodiment, the invention provides a system of introducing a particle beam for low contaminate processing. The system includes a particle accelerator apparatus configured to generate a first particle beam. The system further includes a beam filter coupled to the linear accelerator apparatus to receive the first particle beam. The beam filter is in a first chamber and is configured to generate a second particle beam with substantially a first ionic specie. Moreover, the system includes an end-station with a second chamber coupled to the first chamber for extracting the second particle beam. The second particle beam is irradiated onto a planar surface of a workpiece loaded in the second chamber for implanting the first ionic specie.

In yet another specific embodiment, the present invention provides a method of introducing an accelerator particle beam for low contaminate processing. The method includes generating a plurality of ionic particles by an ion source. The plurality of ionic particles contain multiple species including a first ion specie. Additionally, the method includes accelerating and confining the plurality of ionic particles to a first particle beam with energy level of the first specie at least in a range of about 0.5 MeV to 5 MeV and greater, for example 10 MeV using a particle accelerator. The method further includes extracting the first particle beam through a first exit aperture to a first chamber. Moreover, the method includes processing the first particle beam in the first chamber to separate the first ion specie out of the multiple species and extracting a second particle beam through a second exit aperture into a second chamber. The second particle beam includes substantially the first ion specie only. Furthermore, the method includes irradiating the second particle beam to one or more planar surfaces of one or more workpieces loaded in the second chamber to implant the first ion specie. In one embodiment, the first chamber is associated with a first pressure and the second chamber is associated with a second pressure that is higher than the first pressure.

Numerous benefits are achieved using embodiments of the present invention. In particular, certain embodiments of the present invention may use a linear accelerator based on RFQ-linac and/or RFI technology that has been proven to be a cost effective way to obtain high-energy proton beam in 1 MeV to 5 MeV or higher. Alternative embodiments may employ a cyclotron particle accelerator. Alternative embodiments may employ other types of particle accelerators such as a DC electrostatic accelerator, an example of which is the DYNAMITRON proton accelerator available from Ion Beam Applications SA, Belgium) can also be used. Other DC electrostatic accelerators which may be used include Van de Graaff or Tandem Van de Graaff types. According to certain embodiments of the invention with proper dosage and temperature controls these high-energy H⁺ ions can be utilized for deep implantation down to 200 μm beneath a surface of a selected bulk semiconductor with minimum surface damage to form a desired cleave region thereof. Subsequently, through various controlled cleaving processes or direct layer transfer processes a free-standing thick film (with thickness about 200 μm or less) can be produced. Some embodiments of the invention can be used to produce free-standing single crystalline silicon or polycrystalline silicon thick films for manufacture photovoltaic cells. For example, implanting H+ ions at 5 MeV into silicon, would generate an approximate cleave depth of 220 μm. Some other embodiments of the present invention provide a method of introducing high energy particles for ion implantation with a less contaminate process. The method utilizes a beam filter to separate a desired ionic specie, for example, the H⁺ ion, from other contaminate species with different mass or charge which may be originated from the ion source and generated during propagation through the particle accelerator. Therefore, less contaminate ions remain in the particle beams the subsequent implantation. Those contaminates, if being implanted, otherwise may generate recombination centers in the target material and wide-spreading defects, instead of forming the cleave region as a predominant 2-D defect network. The contamination-induced recombination centers are of particular concern since these can severely degrade solar cell conversion efficiency. Additionally, the beam filter according to certain embodiments of the invention can bend the beam angle, either horizontally or vertically, providing a geometric flexibility for system arrangement. Particularly, the end-station is such a system can be easily incorporated into a cluster processing tool. Some specific embodiments of the present invention also provide method of performing ion implantation with a less contaminate process by setting a pressure difference between the end-station chamber and the beam filter chamber so that any impurity atoms or molecules sputtered by the particle beam can be prevented from re-depositing onto the implanting surface. Depending upon the embodiment, one or more of these benefits may be achieved. These and other benefits may be described throughout the present specification and more particularly below.

Embodiments in accordance with the present invention achieve these benefits and others in the context of known process technology. However, a further understanding of the nature and advantages of the present invention may be realized by reference to the latter portions of the specification and attached drawings.

### BRIEF DESCRIPTION OF THE DRAWINGS

FIG. 1 is a simplified diagram illustrating an acceleration apparatus for introducing charged particle beams for manufacture of a detachable free-standing film of semiconductor materials according to an embodiment of the present invention;

FIG. 2A is a top view of a simplified diagram illustrating a system for introducing low contaminate charged particle beams for manufacture of a detachable free-standing film according to an embodiment of the present invention;

FIG. 2B is a top view of a simplified diagram illustrating a system for introducing low contaminate charged particle beams for manufacture of a detachable free-standing film according to another embodiment of the present invention;

FIG. 3A is a side view of a simplified diagram illustrating a system for introducing low contaminate charged particle beams for manufacture of a detachable free-standing film according to an alternative embodiment of the present invention;

FIG. 3B is a side view of a simplified diagram illustrating a system for introducing low contaminate charged particle beams for manufacture of a detachable free-standing film according to another alternative embodiment of the present invention;

FIG. 4 is a simplified diagram illustrating a method of introducing linear accelerator particle beam for low contaminate processing according to an alternative specific embodiment of the present invention.

FIG. 5 is a simplified schematic diagram illustrating components of an embodiment of an apparatus for performing implantation according to the present invention.

FIG. 5A shows an enlarged schematic view of the ion source and low energy beam transport section of the apparatus of FIG. 5.

FIG. 5B shows an enlarged schematic view of the scanning device of the apparatus of FIG. 5.

FIG. 6 is a schematic illustration of a computer system for use in accordance with embodiments of the present invention.

FIG. 6A is an illustration of basic subsystems the computer system of FIG. 6.

FIGS. 7A-B show simplified cross-sectional views of a controlled cleaving process according to an embodiment of the present invention.

### DETAILED DESCRIPTION OF THE INVENTION

Embodiments in accordance with the present invention relate generally to techniques including a method and a structure for forming substrates using layer transfer. More particularly, the present invention provides a method and system using a linear accelerator particle beam with low contaminate process for the manufacture of thick free-standing semiconductor films for a variety of applications including photovoltaic cells. But it will be recognized that the invention has a wider range of applicability; it can also be applied to other types of applications such as for three-dimensional packaging of integrated semiconductor devices, photonic or optoelectronic devices, piezoelectronic devices, flat panel displays, microelectromechanical systems (“MEMS”), nano-technology structures, sensors, actuators, integrated circuits, biological and biomedical devices, and the like.

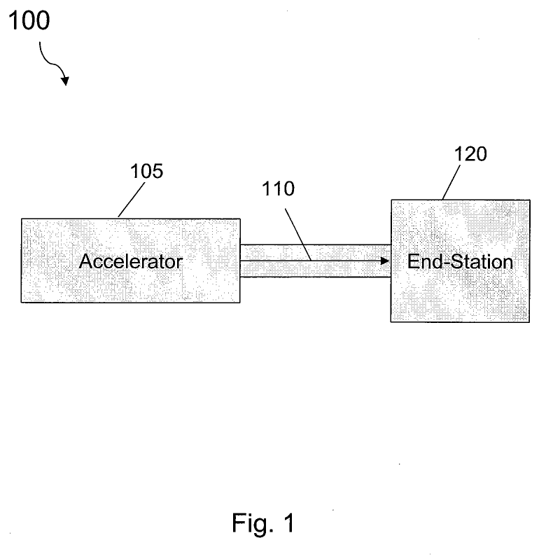

FIG. 1 is a simplified diagram illustrating a system using an apparatus such as a linear acceleration apparatus for introducing charged particle beams for manufacture of a detachable free-standing film of semiconductor materials according to an embodiment of the present invention. This diagram is merely an example, which should not unduly limit the scope of the claims recited herein. One of ordinary skill in the art would recognize many variations, modification, and alternatives. As shown, the system 100 includes a linear accelerator apparatus 105 introducing an ionic particle beam 110 which is guided to an end-station 120 where the particle beam 110 may be used for implanting the ionic particles into a surface of a pre-loaded workpiece. Depending on applications, the workpiece can be a solid film coating on a substrate, a tile material, a bare or preprocessed wafer, or a bulk material. In one specific embodiment, the workpiece is a bulk semiconductor material including ingot of single crystal silicon or polycrystalline silicon. Depending on specific particle species, the energy level of the beams, implantation dose, temperature near the surface region, and other process conditions as well, the ionic particles can penetrate down to a certain depth beneath the surface of the workpiece and cause some local structure damages or form a defect network layer predominantly near the end-of-range (EOR). The defect network around the EOR depth then can be utilized for producing a free-standing thick film through an isothermal thermally-induced separation process, controlled cleaving process, either bonded or using a direct film transfer process. In one embodiment, free-standing single crystal silicon thick films or tiles can be produced out of bulk ingot in a proprietary Direct Film Transfer process, for example as described in U.S. Provisional Patent Application No. 60/886,827, incorporated by reference herein, very cost-effectively and then may be used for manufacture of photovoltaic cells or for other applications.

Using hydrogen as the implanted species into the silicon wafer for photovoltaic application as an example, the desired implantation dose ranges from about 1×10¹⁵ to about 1×10¹⁶ atoms/cm², and preferably the dose is less than about 5×10¹⁶ atoms/cm². Using a suitable hydrogen plasma source such as an electron cyclotron resonance (ECR) ion source and acceleration extractor voltage, the hydrogen gas is ionized in a microwave plasma and a beam of H⁺ ions is formed by using a low-voltage particle extractor (<100 kV) and then shaped by suitable beam optics such as an Einzel lens or solenoid. Eventually, the linear accelerator apparatus can accelerate the H⁺ ion beam to an energy level ranging from about 0.5 MeV and greater to about 5 MeV and greater. The H⁺ ion beam is guided into the end-station where one or more silicon ingots may be ready for the implantation process. Depending on certain embodiments, implantation temperature ranges from about −50 to about 550 Degrees Celsius is selected, and is preferably less than about 400 Degrees Celsius to prevent a possibility of H⁺ ions from diffusing out of the implanted silicon ingot. According to certain preferred embodiments, the H⁺ ions in an energy level ranging from 1 MeV to 5 MeV or greater are implanted with about 1×10¹⁶ atoms/cm² dose. The implantation process can have multiple steps with various dosage for each step. The implanted H⁺ ions causes the formation of a cleavable region at a depth ranging from approximately 10 μm (corresponding to an implant energy of about 750 keV) to over 120 μm or even 150 μm (corresponding to implant energies of 3.5 MeV and 4 MeV respectively) beneath the silicon surface. After that, further processes can be performed including a controlled cleaving process to produce a thick silicon free-standing film with a thickness μm to over 120 μm, which further can be used for the manufacture of photovoltaic cell.

In a specific embodiment, the present method uses a linear accelerator apparatus based on Radio-Frequency Quadrupole linear accelerator (RFQ-Linac) or Drift-Tube Linac (DTL), Quadrupole-Focused Interditigated Linac (QFI), or RF-Focused Interdigitated (RFI) technology. Some or all of the above technologies are available from companies such as Accsys Technology Inc. of Pleasanton, Calif.; Linac Systems, LLC of Albuquerque, N. Mex. 87109, and others. Alternatively, a type of accelerator apparatus which may be utilized is a DC electrostatic accelerator such as the DYNAMITRON mentioned above.

And while the particular embodiment described above utilizes a linear accelerator apparatus, this is not required by the present invention. In alternative embodiments, other types of particle accelerators, such as cyclotron or the like, can be used. An example of such a cyclotron particle accelerator which may be used is the CYCLONE 10/5 or 18/9 also available from Ion Beam Applications SA.

In another specific embodiment, these approaches use RF acceleration of an ion beam extracted from an ion source (such as an ECR ion source), to increase the total energy of the H⁺ ion beam from a range of approximately 20-100 keV to 0.5 to 7 MeV or more. The H⁺ ion current available with these technologies can be up to 100 mA or more. As a specific example, assuming 100 kW of beam power, a 3.5 MeV RFQ/RFI-Linac would yield a proton beam current of about 29 mA. Using a dose of approximately 1×10¹⁶ ion/cm² and an expanded beam of about 500 mm×500 mm, the area per hour is about 6.5 square meters while the power flux is kept to about 40 Watts/cm².

Alternatively, the beam can be spatially averaged using a fast scanning method such as using a electro-magnetic beam scanning system, to take a smaller, non-expanded beam quickly “paint” the target area. This approach effectively averages the high-power beam flux.

Such a configuration may offer concurrent advantages and further system flexibility. For example, electro-magnetic beam scanning can lower the throughput losses and decrease the overall system size, by keeping the beam overscan to a smaller percentage. Specifically, since overscan is a function of the beam diameter, an expanded beam would need a larger overscan which lowers throughput and causes more X-Y scan travel. This configuration also reduces system size and allows the possibility of implant dose patterning under scan control.

While certain embodiments have been described in connection with an ECR ion source, the present invention is not limited to this particular example. In accordance with alternative embodiments, ions can be extracted from other sources, including but not limited to magnetized or non-magnetized microwave sources, and hot cathode ion sources.

Of course, there can be other variations, alternatives, and modifications. For example, the high-energy H⁺ ion beam based on the above combination of parameters may be applied in a Direct Film Transfer (DFT) process for producing free-standing thick silicon films out of the bulk silicon ingot, making it a particularly cost-effective way to manufacture solar cells.

However, due to the imperfection of the ion source, in one embodiment, other contaminate species in addition to the desired ionic specie (e.g., H⁺ ion) may be included. All these ionic species will be accelerated by the linear accelerator into the ionic particle beam such as the beam 110. In another embodiment, many unavoidable contaminants may be generated during the acceleration process along the path while all particles propagate through the linear accelerator into the end-station. For example, ionic or neutral species like Hydrogen isotope species, Helium species, Oxygen species, Nitrogen species, or Carbon species, sputtered metals such as Aluminum, Iron, Manganese, Magnesium, Copper and Tungsten, etc may be included into the beam 110 as contaminates. Some of these contaminate ion species may degrade the formation of a clean two dimensional EOR region and its overlying film for cleaving in the Direct Film Transfer process mentioned above. The desired H⁺ ion beam is required to be separated out of other contaminate species for a desired low contaminate implantation process. According to certain embodiments of the present invention, a beam filtering mechanism is used to reduce the impurity levels of the high-energy particle beam produced by the linear accelerator and implement for a low contaminate Direct Film Transfer process for solar cell production. Of course, the filtered ionic particle beam with substantially the desired ion specie may have much wider ranges of applications.

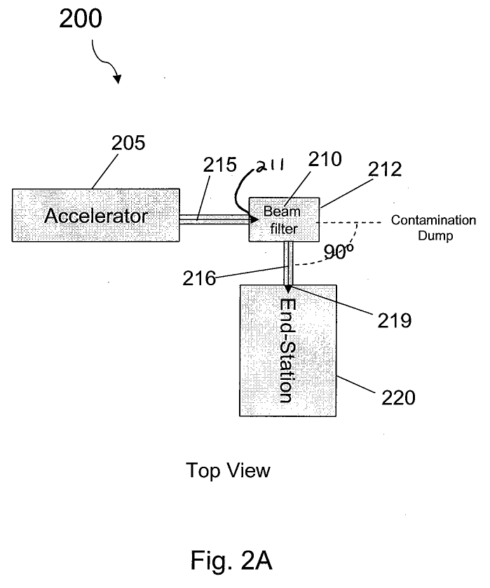

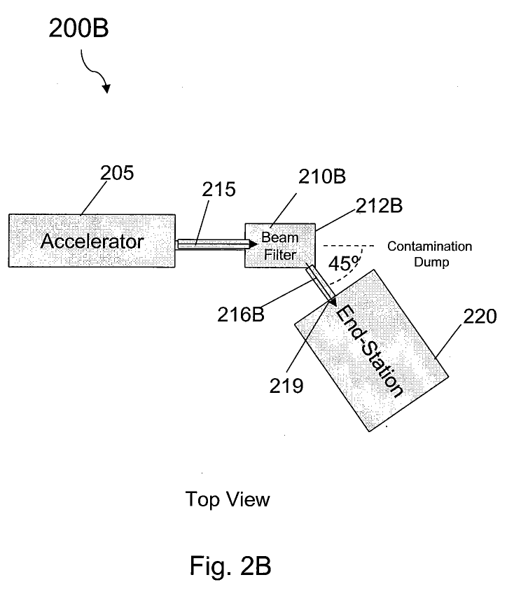

FIG. 2A is a top view of a simplified diagram illustrating a system for introducing low contaminate charged particle beams for manufacture of a detachable free-standing film o according to an embodiment of the present invention. This diagram is merely an example, which should not unduly limit the scope of the claims recited herein. One of ordinary skill in the art would recognize many variations, modification, and alternatives.

As shown in FIG. 2A, the system 200 comprises a linear accelerator apparatus 205, a beam filter 210 configured to receive a first particle beam 215 through a first aperture 211 and output a second particle beam 216 through a second exit aperture 219, and an end-station 220 configured to receive the extracted second particle beam 216 for processing with a workpiece pre-loaded into the end-station. In one embodiment, the linear accelerator apparatus 205 is linear accelerator apparatus 105 based on RFQ-linac or RFI technology or the like. Alternative embodiments may employ a DC electrostatic accelerator or a cyclotron or the like. In another embodiment, the particle beam 215 includes one major ion specie in addition to some traceable other impurity. The particle beam 216 after the beam filter 210 includes substantially only the major specie with other impurity content much reduced. For example, the major ion specie is H⁺ ion (protons).

In one embodiment, the beam filter 210 is configured to process all the ionic species contained in the first particle beam 215. In one specific embodiment, the beam filter 210 includes a mass-selection device based on a mechanism of generating separate trajectories with different radius of curvature for particles with different charge-to-mass ratio. For example, as a high-energy ionic particle with mass heavier than the desired first ionic specie is received by the beam filter, its trajectory may be curved with a larger radius than that of the first ion specie. The mass-selection device includes an internal passage channel to match with the curved trajectory of the first ion specie. The mass-selection device also includes one or more ion traps set in the trajectories of unwanted ion species. Therefore, the desired first ionic specie can be separated out and be extracted as the second particle beam while the rest of un-desired ionic species may be dumped. For example, the beam filter 210 outputs a second particle beam 216 with nearly pure H⁺ ions while dumps all other heavier ions and neutral species elsewhere. Then the filtered second particle beam can be utilized for various material processing applications with a low contaminate process.

In one embodiment, an electric sector may be used for fulfilling the above mass-selection for the charged particles. In another embodiment, the beam filter may include a magnetic sector for performing the mass-selection. In yet another embodiment, various types of combination of one or more electric sectors, one or more magnetic sectors, and one or more drift channels can be assembled to build the beam filter.

As a result of the above mass-selection process based on a mechanism for differentiating charge-to-mass ratio, the spatial direction of the second particle beam is different from that of the first particle beam received by the mass-selection device, forming an effective beam-bending angle from a first direction to a second direction. For example, the beam filter is designed to allow the second particle beam including the desired H⁺ ions being extracted in a direction forming an angle of 90 degree relative to original beam direction. Other heavier ionic specie cannot reach the exit aperture due to their different trajectories and are dumped to certain ion traps set aside.

Depending on the applications, the beam filter can be designed to have different effective beam-bending angles for different desired ion species. For example, the beam filter may have a first passage channel for H⁺ ions for one application/process and a second passage channel for H₂⁺ ions for another application/process. Of course, there can be other variations, alternatives, and modifications. For example, the charge-to-mass ratio mechanism may be utilized for the separation of like charged particles of different energies.

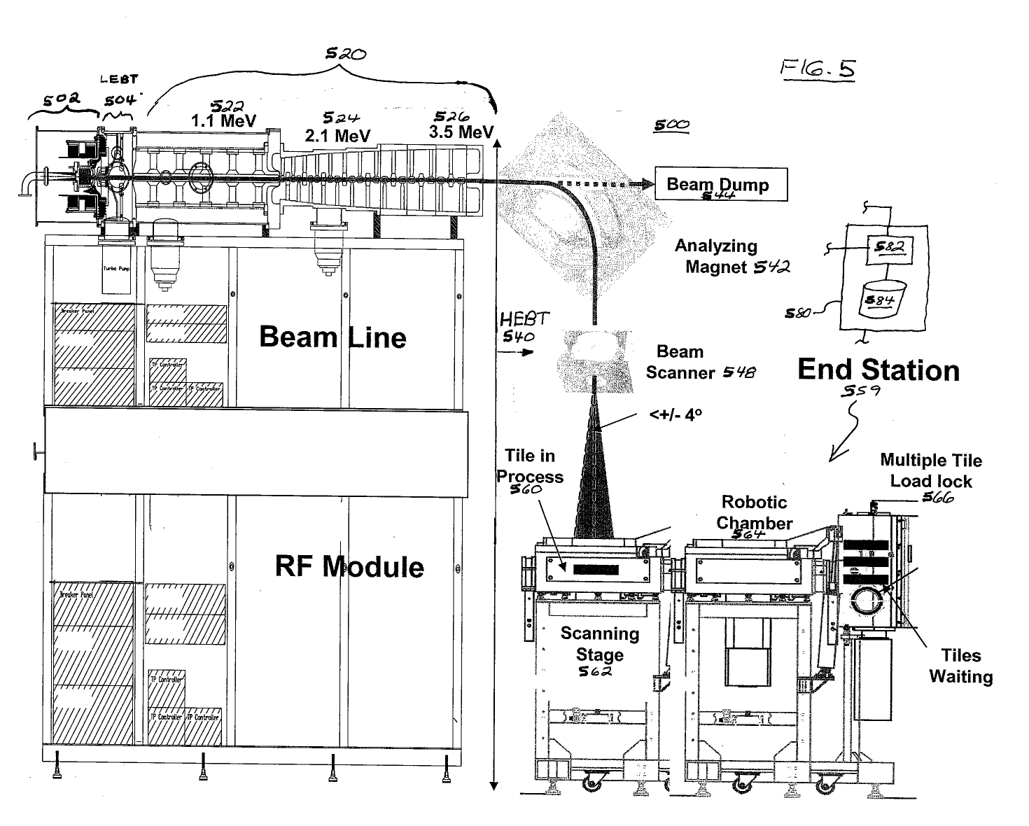

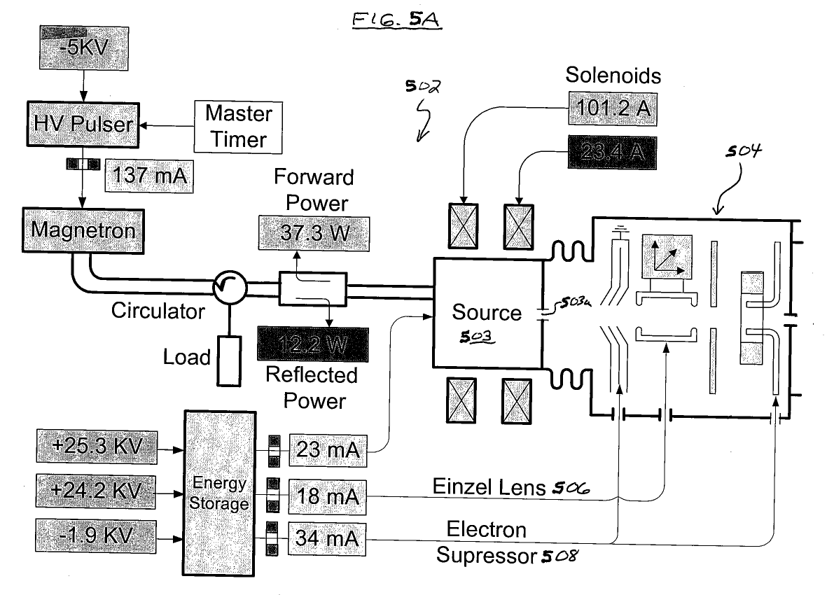

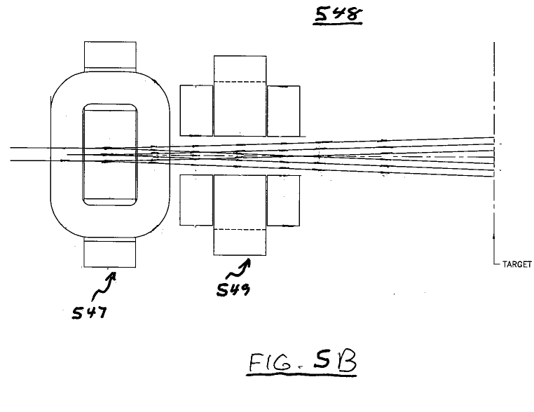

As mentioned above, certain embodiments in accordance with the present invention may employ a scanning mode for implantation. An example of such an embodiment is shown in the simplified schematic views of FIGS. 5-5A. In particular, FIG. 5 is a simplified schematic diagram illustrating components of an embodiment of an apparatus for performing implantation according to the present invention. FIG. 5A shows an enlarged schematic view of the ion source and low energy beam transport section of the apparatus of FIG. 5.

Apparatus 500 comprises ion source 502 in vacuum communication with Low Energy Beam Transport (LEBT) section 504. The LEBT section 504 performs at least the following functions.

Referring to FIG. 5A, the LEBT takes the ions that stream out of the aperture 503a in the ion source chamber 503, and accelerates these ions to a relatively low energy (100 keV or less, and here ˜30 keV). This acceleration of the ions achieves the resonance velocity necessary to couple synchronously to the first, Radio Frequency Quadrupole (RFP) stage 522 of the succeeding linear accelerator (linac) section 520.

Examples of ion sources include ECR ion sources, magnetron ion sources, and Penning sources. Examples of ionization methods include the use of e-beams, lasers, glow discharge, and thermal techniques.

The LEBT 504 also typically functions to shape the ion beam for optimum acceptance into the first, RFP stage 522 of the linac section 520. In this particular embodiment, the beam shaping element is an Einzel lens 506.

The LEBT 504 also include an electron suppressor element 508. This element 508 serves to suppress secondary electrons generated by errant ions interacting with exposed surfaces of the LEBT.

Upon entry into the linac section 520, the ion beam is accelerated to higher and higher energies by successive stages. In the first, RFP stage 522, the ions are accelerated from the energy of ˜30 keV, to an energy of about 1.1 MeV. In a second linac stage 524, the ions are accelerated to about 2.1 MeV. In the third and final linac stage 526, the ions are accelerated to energies of about 3.5 MeV or even greater.

Upon exiting the linac section 520, the ion beam enters the High Energy Beam Transport (HEBT) section 540. The function of the HEBT section 540 is to shape the highly energetic ion beam exiting from the final linac stage 526 (e.g. from elliptical to circular), to bend the path of the highly energetic ion beam, and, if appropriate, to achieve scanning of the beam on the target.

Specifically, the highly energized ion beam is first exposed to analyzing magnet 542, which alters the direction of the beam and performs the cleansing function described throughout the instant application, such that initial contaminants of the high energy beam are routed to beam dump 544.

In accordance with certain embodiments, the analyzing magnet 542 exerts a force over the beam that is consistent over time, such that the resulting direction of the of the cleansed beam does not vary. In accordance with alternative embodiments, however, the analyzing magnet may exert a force over the beam that does change over time, such that the direction of the beam does in fact vary. As described in detail below, such a change in beam direction accomplished by the analyzing magnet, may serve to accomplish the desired scanning of the beam along one axis.

Upon exiting the analyzing magnet, the cleansed ion beam enters beam scanner 548. FIG. 5B shows a simplified schematic diagram of one embodiment of the beam scanner 548 in accordance with the present invention. Specifically, beam scanner 548 comprises a first scanner dipole 547 configured to scan to vary the location of the beam in a first plane. Beam scanner 548 also comprises a second scanner dipole 549 configured to scan to vary the location of the beam in a second plane perpendicular to the first plane.

While the particular embodiment of the beam scanner shown in FIG. 5B includes two dipoles, this is not required by the present invention. In accordance with alternative embodiments, the beam scanner could include only a single dipole. Specifically, in accordance with certain embodiments, the analyzer magnet located upstream of the beam scanner, could be utilized to provide scanning in a plane perpendicular to that in which scanning is achieved by a single dipole of the beam scanner. In one such embodiment, time-variance in the magnetic field of the analyzer magnet may result in an energized beam whose direction varies by +/−4° from the normal. Such “wobble” in the direction of the cleansed beam exiting the analyzing magnet, may be utilized for scanning in place of a second dipole of the beam scanner. Alternatively, such a wobbled beam may be used in conjunction with a beam scanner also having a second dipole, such that magnitude of scanning in the direction of the wobble is increased.

While the particular embodiment shown in FIG. 5 includes elements for shaping and controlling the path of the beam, these are not required by the present invention. Alternative embodiments in accordance with the present invention could employ a drift tube configuration, lacking such elements and allowing the shape of the beam to expand after it exits the accelerator.

FIG. 5 shows the remaining components of the apparatus, including an end station 559. In this end station 559, tiles 560 in the process of being scanned with the energetic ion beam, are supported in a vacuum in scanning stage 562. The tiles 560 are provided to the scanning stage through a robotic chamber 564 and a load lock 566.

The scanning stage 562 may function to translate the position of the workpieces or bulk materials receiving the particle beam. In accordance with certain embodiments, the scanning stage may be configured to move along a single axis only. In accordance with still other embodiments, the scanning stage may be configured to move along two axes. As shown in the particular embodiment of FIG. 5, physical translation of the target material by the scanning stage may be accompanied by scanning of the beam by the scanning device acting alone, or in combination with scanning accomplished by the beam filter. A scanning stage is not required by the present invention, and in certain embodiments the workpieces may be supported in a stationary manner while being exposed to the radiation.

The various components of the apparatus of FIGS. 5-5B are typically under the control of a host computer 580 including a processor 582 and a computer readable storage medium 584. Specifically, the processor is configured to be in electronic communication with the different elements of the apparatus 500, including the ion source, accelerator, LEBT, HEBT, and end station. The computer readable storage medium has stored thereon codes for instructing the operation of any of these various components. Examples of aspects of the process that may be controlled by instructions received from a processor include, but are not limited to, pressures within the various components such as end station and the HEBT, beam current, beam shape, scan patterns (either by scanning the beam utilizing a scanner and/or analyzing magnet, and/or moving the target utilizing translation with XY motored stages at substrate, i.e. painting), beam timing, the feeding of tiles into/out of the end station, operation of the beam cleaning apparatus (i.e. the analyzing magnet), and flows of gases and/or power applied to the ion source, etc.

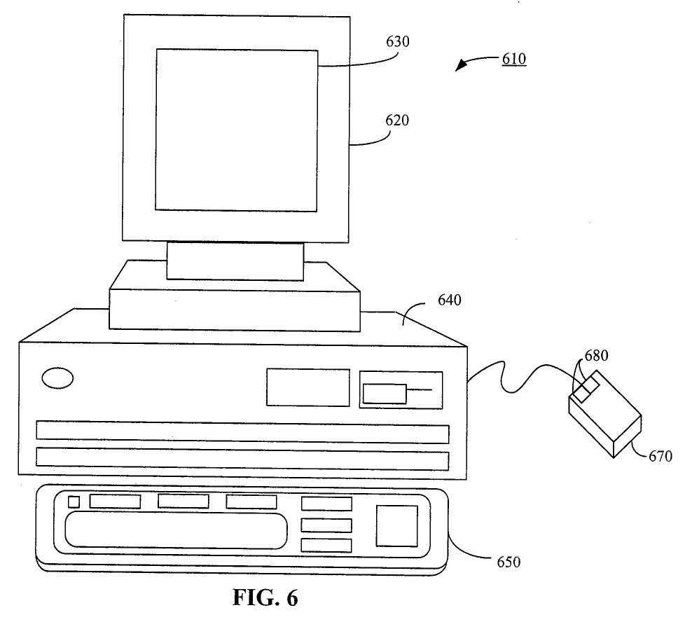

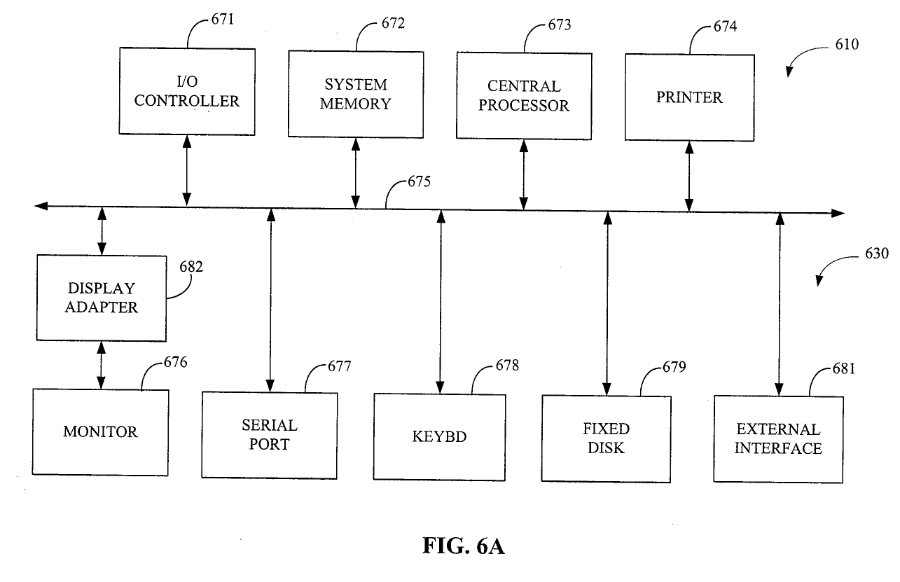

The various components of the coupon system described above may be implemented with a computer system having various features. FIG. 6 shows an example of a generic computer system 610 including display device 620, display screen 630, cabinet 640, keyboard 650, and mouse 670. Mouse 670 and keyboard 650 are representative “user input devices.” Mouse 670 includes buttons 680 for selection of buttons on a graphical user interface device. Other examples of user input devices are a touch screen, light pen, track ball, data glove, microphone, and so forth. FIG. 6 is representative of but one type of system for embodying the present invention. It will be readily apparent to one of ordinary skill in the art that many system types and configurations are suitable for use in conjunction with the present invention. In a preferred embodiment, computer system 610 includes a Pentium class based computer, running Windows NT operating system by Microsoft Corporation. However, the apparatus is easily adapted to other operating systems and architectures by those of ordinary skill in the art without departing from the scope of the present invention.

As noted, mouse 670 can have one or more buttons such as buttons 680. Cabinet 640 houses familiar computer components such as disk drives, a processor, storage device, etc. Storage devices include, but are not limited to, disk drives, magnetic tape, solid state memory, bubble memory, etc. Cabinet 640 can include additional hardware such as input/output (I/O) interface cards for connecting computer system 610 to external devices external storage, other computers or additional peripherals, further described below.

FIG. 6A is an illustration of basic subsystems in computer system 610 of FIG. 6. This diagram is merely an illustration and should not limit the scope of the claims herein. One of ordinary skill in the art will recognize other variations, modifications, and alternatives. In certain embodiments, the subsystems are interconnected via a system bus 675. Additional subsystems such as a printer 674, keyboard 678, fixed disk 679, monitor 676, which is coupled to display adapter 682, and others are shown. Peripherals and input/output (I/O) devices, which couple to I/O controller 671, can be connected to the computer system by any number of means known in the art, such as serial port 677. For example, serial port 677 can be used to connect the computer system to a modem 681, which in turn connects to a wide area network such as the Internet, a mouse input device, or a scanner. The interconnection via system bus allows central processor 673 to communicate with each subsystem and to control the execution of instructions from system memory 672 or the fixed disk 679, as well as the exchange of information between subsystems. Other arrangements of subsystems and interconnections are readily achievable by those of ordinary skill in the art. System memory, and the fixed disk are examples of tangible media for storage of computer programs, other types of tangible media include floppy disks, removable hard disks, optical storage media such as CD-ROMS and bar codes, and semiconductor memories such as flash memory, read-only-memories (ROM), and battery backed memory.

Any of the software components or functions described in this application, may be implemented as software code to be executed by a processor using any suitable computer language such as, for example, Java, C++ or Perl using, for example, conventional or object-oriented techniques. The software code may be stored as a series of instructions, or commands on a computer readable medium, such as a random access memory (RAM), a read only memory (ROM), a magnetic medium such as a hard-drive or a floppy disk, or an optical medium such as a CD-ROM. Any such computer readable medium may reside on or within a single computational apparatus, and may be present on or within different computational apparatuses within a system or network.

FIG. 2B is a top view of a simplified diagram illustrating an alternative embodiment of a system for introducing low contaminate charged particle beam according to a specific embodiment of the present invention. This diagram is merely an example, which should not unduly limit the scope of the claims recited herein. One of ordinary skill in the art would recognize many variations, modification, and alternatives. As shown the beam filter 210B changes spatial direction of the H⁺ ion beam generated by the linear accelerator 205 by a 45-degree angle, compared to the beam filter 210 shown in FIG. 2A which extracts the H⁺ ion beam in a 90-degree angle relative to first particle beam direction.

In one embodiment, the beam filter, although changing the beam direction, causes little energy change of the energy level of the selected first ion specie to form the second beam 216. In other words, the energy level of particle beam 216 may still in the desired range of 1 MeV to 5 MeV obtained by the linear accelerator 205. Of course, there can be other variations, modifications, or alternatives. For example, the energy level and beam confinement level of the first particle beam 215 may affect the beam profile of the output second particle beam 216. Also, the particle accelerator could be of a type other than a linear accelerator, such as a cyclotron or the like.

In yet another specific embodiment, the beam filter 210 or 210B respectively is installed in a vacuum chamber 212 or 212B. The chamber 212 or 212B has an independent pumping unit (not shown) which may control the internal pressure to a certain value of P_{bf} during the operation. The chamber 212 or 212B also includes a high-energy beam transport unit at the exit aperture 219 for extracting the particle beam 216. The High Energy Beam Transport (HEBT) includes the entire beam path from the exit of 205 to the entrance of 220 or exit aperture 219. The exit aperture 219 is directly coupled to end-station 220 which includes a process chamber for performing a high-energy implantation process. In one embodiment, the process chamber includes its own pumping unit to control its own internal pressure of another predetermined level of Pₑₛ. Of course, there can be other variations, alternatives, and modifications.

As an example, the high-energy charged particle beam extracted at the exit 219 is a H⁺ ion beam. In the end-station 220 the H⁺ ion beam are guided onto the surface of the workpiece. While the impinging high-energy H⁺ ions predominantly penetrate into some distances into the bulk, some surface atoms or molecules, especially, some light weighted surface contaminates with higher vapor pressure, may be sputtered away by the high-energy H⁺ ions. These sputtered surface atoms or molecules may redeposit on the surface of the workpiece causing damages or increasing surface roughness. The surface contaminate re-deposition may alter the continued ion implantation process resulting a domino effect that lead to poor non-uniform cleaving planes not suit for subsequent film transfer process. In a preferred embodiment, in order to prevent the sputtered surface atoms or molecules from re-depositing on the processed surface of the workpiece, the pressure level P_{bf} of the beam filter chamber 212 or 212B is intentionally set to be lower than the pressure level Pₑₛ of the end-station chamber 220. Because of the pressure difference, the sputtered contaminate atoms or molecules can be pulled backward due to the negative differential pressure to the beam filter chamber away from, instead of being deposited to, the surface of the workpiece. The desired pressure difference may depend on the ion beam energy level, beam profile, or scanning mechanism, and material types and surface conditions of the workpiece. For example, a typical pressure Pₑₛ of the chamber at the end-station 220 could be less than about 1×10⁻³ Torr. The chamber pressure P_{bf} that houses the beam filter 210 then can be set to be one or two order magnitude lower than the pressure Pₑₛ so that the sputtered contaminates may be sucked away. This arrangement also tends to keep any neutral or sputtered atoms generated within the accelerator or high-energy beam transport prior to the analyzing beam filter away from the end-station. Of course, there can be other variations, alternatives, and modifications.

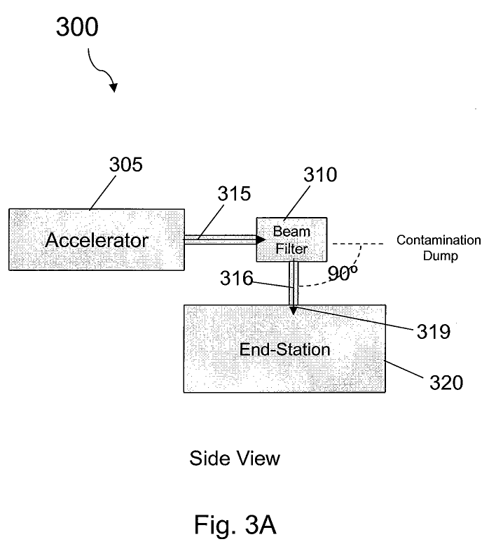

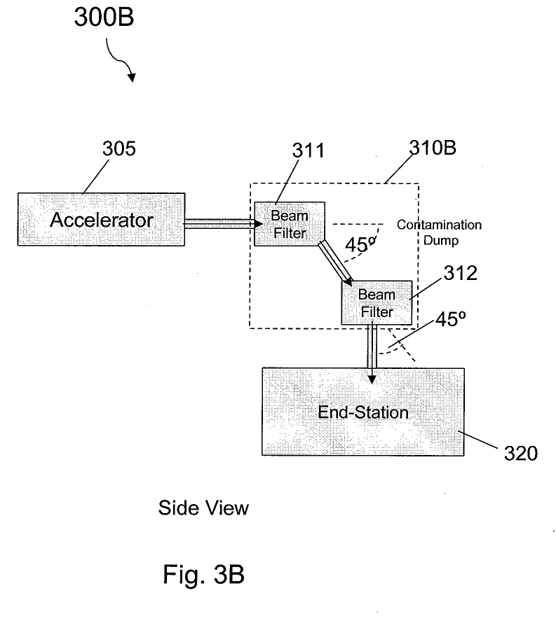

FIG. 3A is a side view of a simplified diagram illustrating a system for introducing low contaminate charged particle beams for manufacture of a detachable free-standing film of semiconductor materials according to an alternative embodiment of the present invention. This diagram is merely an example, which should not unduly limit the scope of the claims recited herein. One of ordinary skill in the art would recognize many variations, modification, and alternatives.

As shown in FIG. 3A, the system 300 comprises a linear accelerator apparatus 305 configured to generate a first particle beam 315 in a horizontal direction. In one embodiment, the linear accelerator 305 is substantially similar to the linear accelerator 205. The first particle beam 315 is substantially the same as the particle beam 215. Additionally, the system 300 includes a mass-selection beam filter 310 configured to receive a first particle beam 315 and output a second particle beam 316 in a vertical downward direction, i.e., ninety degree relative to the first particle beam 315 within a vertical plane. Similar to the function of the beam filter 210, the beam filter 310 is able to separate a major ion specie out of the first particle beam to have the second particle beam with substantially only the desired major ion specie. In a specific embodiment, the beam filter 310 includes a first chamber that houses a mass-selection device that comprising one or more electric/magnetic sectors and certain passage channel for extracting the second particle beam with selected ion specie. The first chamber should be in a vacuum environment. Furthermore, the system 300 includes an end-station 320 configured to receive the second particle beam 316 for implanting the desired ionic particles into one or more pre-loaded workpiece.

If beam scanning over the target material is desired, in particular embodiments an electromagnetic X-Y type scanning system can be added to the output side of the beam filter assembly. One of the scanning axes can also be combined with the analyzing beam filter system to “wobble” or add/subtract an angle from the last stage of the beam filter, in order to effectuate the scanning along that specific axis.

In an specific embodiment, the end-station includes a second chamber that is part of a cluster tool (not shown) For example, the cluster tool may include an open-air tile-staging station for loading multiple bulk workpieces. The cluster tool may include one more load locks that are directly coupled to the second chamber for transferring the workpiece in/out of the second chamber from/to atmosphere environment or in/out of different chambers with different processing pressures. In one embodiment, the workpiece is first loaded on to a rack with a proper clamping mechanism. The workpiece can be various types including but not limiting to a film coating on a substrate, a tile, a wafer, and a bulk material with a planar surface. In another embodiment, a plurality of bulk workpieces can be loaded on the rack and packed in certain order. Then the loaded rack with a plurality of bulk workpieces is transferred into the second chamber for batch processing. Through one or more load locks other process stations/chambers may be included/added for the cluster tool. For example, there is a process chamber for polishing or re-lapping the surfaces of the bulk workpieces before or after ion implantation process. In another example, a rack of loaded bulk workpieces can be annealed or inspected before a cleaving process in a post-processing chamber after the high-energy implantation. Furthermore, another process chamber can be coupled to the post-processing chamber for completing a controlled cleaving process to manufacture free-standing thick films or tiles out of those bulk workpieces. After one layer film or tile is removed, the remaining bulk workpieces on the rack will be transferred back to the second chamber of the end-station in the cluster tool for further ion implantation as a cycled direct film transfer process. Of course, there can be other variations, alternatives, and modifications.

FIG. 3B is a side view of a simplified diagram illustrating a system for introducing low contaminate charged particle beam according to another alternative embodiment of the present invention. This diagram is merely an example, which should not unduly limit the scope of the claims recited herein. One of ordinary skill in the art would recognize many variations, modification, and alternatives. As shown, the system 300B includes the linear accelerator apparatus 305 and a two-stage beam filter 310B that are configured in a vertical plane similar to the system 300. In one embodiment, the two-stage beam filter 310B includes just a series combination of two single-stage beam filters 311 and 312 each with an effective 45-degree beam-bending angle. In other words, the two-stage beam filter 310B is configured to have a 90 degree final effective beam-bending angle. Benefits of using two beam filters include cost reduction, improved mass-selectivity, and geometric configuration flexibility for the whole cluster tool.

If beam scanning over the target material is desired, an electromagnetic X-Y type scanning system can be added to the output side of the last beam filter assembly 312. One of the scanning axes can also be combined with the analyzing beam filter system to “wobble” or add/subtract an angle from the last stage of the beam filter to effectuate the scanning along that specific axis. Of course, there can be other variations, alternatives, and modifications.

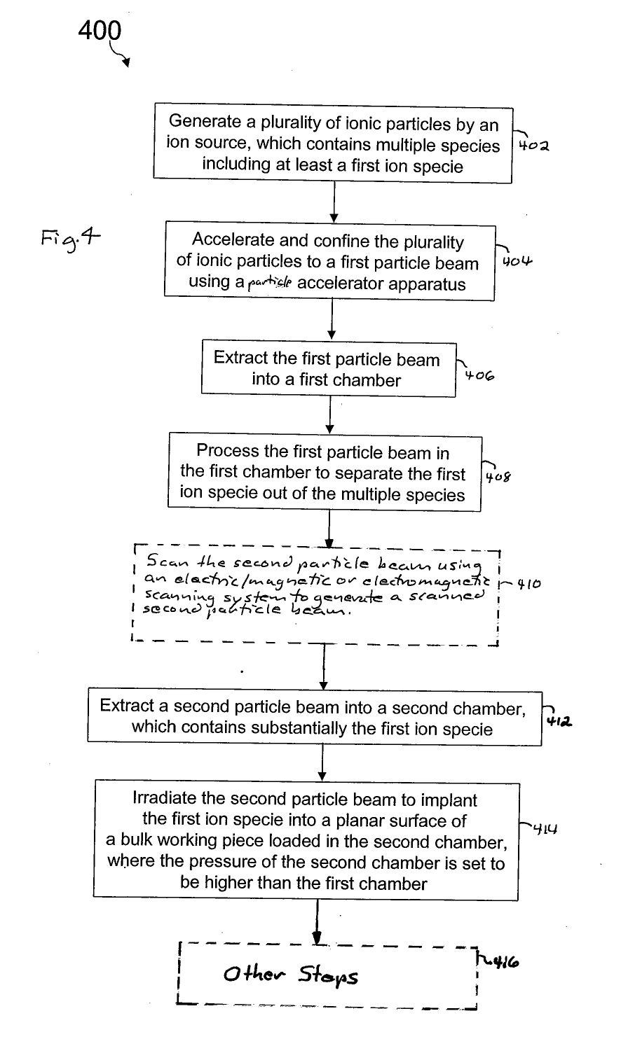

FIG. 4 is simplified diagram illustrating a method of introducing a particle beam such as a linear accelerator particle beam for low contaminate processing according to an alternative specific embodiment of the present invention. As shown, a method 400 of introducing linear accelerator particle beam for low contaminate processing for direct film transferring applications is briefly outlined below.

1. Generate 402 a plurality of ionic particles by an ion source, which contains multiple species including at least a first ion specie (for example H⁺ ions);
2. Accelerate and confine 404 the plurality of ionic particles to a first particle beam with energy level of the first specie at least in a range of 1 MeV to 5 MeV or greater using a particle accelerator apparatus such as a linear accelerator apparatus;
3. Extract 406 the first particle beam through a first exit aperture to a first chamber;
4. Process 408 the first particle beam in the first chamber to separate the first ion specie out of the multiple species;
5. Optionally scan 410 the second particle beam using an electric/magnetic or electro-magnetic scanning system electro-magnetic to generate a scanned second particle beam;
6. Extract 412 a second particle beam through a second exit aperture into a second chamber, which contains substantially the first ion specie (for example H⁺ ions) only;
7. Irradiate 414 the scanned second particle beam to implant the first ion specie into one or more planar surfaces of one or more bulk workpieces loaded in the second chamber, where the pressure of the second chamber is set to be higher than the first chamber;
8. Perform other steps 416, as desired.

The above sequence of steps provides a method according to an embodiment of the present invention. Other alternatives can also be provided where steps are added, one or more steps are removed, or one or more steps are provided in a different sequence without departing from the scope of the claims herein. Alternatively, there can be other ways of reducing contaminate during the high-energy implantation. Further details of the present method can be found throughout the present specification and more particularly below.

As shown in FIG. 4, the method generates a plurality of charged particles using an ion source. For example, the ion source could be an ECR microwave ion source. With supplying of a concentrated hydrogen gas, the generated plurality of charged particles include predominantly protons H⁺ and some isotope ions like D⁺, H⁻, H₂⁺, etc. Of course, there are always some other impurity ionic species due to the imperfection of the ion source and supplied processing gas. Depending on embodiment, H₂⁺ ions may be selected to be main specie so that higher dosage of hydrogen can be achieved for implantation process. In one embodiment, this ion source is just one component of the linear accelerator apparatus 100 shown in FIG. 1. Of course, there can be other variations, modifications, and alternatives.

In one specific embodiment, the plurality of charged particles are accelerated and confined to a charged particle beam by a linear accelerator apparatus. For example, a linear accelerator apparatus based on RFQ-linac technology and/or RFI technology or combination of both can produce a high-energy ionic beam. In other embodiments, the particle accelerator apparatus may comprise a DC electrostatic accelerator or a non-linear accelerator such as a cyclotron. For example, the H+ ions in the beam can be accelerated to an energy level ranging from 1 MeV to 5 MeV or greater. Depending on different ionic species with different mass or charges, the corresponding energy level could vary. Of course, there can be other variations, modifications, and alternatives.

In another specific embodiment, the accelerated particle beam will be firstly extracted through an exit aperture of the linear accelerator apparatus into a first chamber where a beam filter apparatus is installed to receive the particle beam and an input beam. The first chamber should be a vacuum chamber with an independent pumping unit and a gauge for measure the pressure. Again, the input beam includes multiple ionic species, though one specie, e.g., the H⁺ ion may be a dominant specie. The beam filter is designed for separating the dominant ion specie from the total ionic species and dumping the rest impurity species. In one embodiment, the beam filter may be one based on electric sector to separate the H⁺ ion. In another embodiment, the beam filter may be one based on magnetic sector to separate the H⁺ ion. In an alternative embodiment, the mass-selection mechanism may be based on charge-to-mass ratio. In another alternative embodiment, a charge-to-mass ratio may be utilized for specie-selection. Of course, there can be other variations, modifications, and alternatives.

Typically, any combination of electric sectors, electric/magnetic sections (e.g. ExB fields) or magnetic sectors plus certain drift channels may be used to build the beam filter. The charged particle beams, depending on its mass or charges, may be curved to certain angle. In one embodiment, the beam filter is designed to transform the input beam with multiple species to an output beam with only desired ionic specie through an exit aperture of the first chamber while dumping the other species to side channels. The exit aperture of the first chamber may point to a different spatial direction, either horizontally or vertically, than the exit aperture of the linear accelerator apparatus. For example, the output beam may be in a horizontal plane with the input beam but turn 90 degrees away. In another example, the output beam may be in a vertical plane with the input beam and change a 90 degree angle from horizontal direction to downward direction. In another embodiment, because the first chamber is held in a high vacuum environment and the magnetic sector would change little of the speed of the charged particles, the output beam now contains substantially desired ionic species having an energy level substantially the same as the original beam. For example, the output H⁺ ion beam may be still in energy level of 1 MeV and above.

If the accelerator system is to have the capability of generating a range of energy levels for allowing different Direct Film Transfer thicknesses to be made using an implant system, the beam filter can be advantageously made using a electric or electro-magnetic section where the second beam of a different energy can be selected by setting a voltage or current to change the beam bending characteristics of the beam filter. Of course, there can be other variations, modifications, and alternatives.

In yet another embodiment, the output beam is extracted and optionally scanned into a second chamber which is part of an end-station where the charged particles are used for performing various kinds of processes including one or more steps of a Direct Film Transfer process for manufacturing free-standing thick silicon films from ingot for solar cell applications. Typically, the second chamber is held at a high vacuum environment with a pressure of about 5×10⁻⁶ Torr for performing the certain steps of the Direct Film Transfer process. In particular, referring to FIG. 4 again, the method includes irradiating the particle beam to a bulk sample surface for implanting the ionic particle into the surface according to an embodiment of the present invention. Briefly, in a specific embodiment, the particle beam contains substantially pure H⁺ ions with energy in a range of 1 MeV to 5 MeV or greater. These high energy H⁺ ions would be desired for penetrating beneath a bulk surface through deep implantation to form a defect network near its end-of-range (EOR). In certain embodiment, H₂⁺ ions may be used for achieving higher implantation dose. The defect network facilitates a so-called direct film transfer (DFT) process wherein a free-standing thick film can removed from the bulk. This summarizes the so-called Direct Film Transfer process. For example, the Direct Film Transfer process can be applied to produce thick films with thickness ranging in 10 μm to 200 μm out of a bulk monocrystalline, polycrystalline, or amorphous silicon substrate. Additionally, the substrate can be made of III/V materials such as gallium arsenide, gallium nitride (GaN), and others. Direct Film Transfer process provides substantially saving over conventional sawing by reducing the kerf loss over 50%. Furthermore, these thick films can be used produce low cost photovoltaic cells for improved solar energy conversion applications.

In another specific embodiment, during the implantation process, the method 400 provides a process of keeping a pressure difference between the first chamber and the second chamber. In particular, the pressure in the first chamber will be kept lower than the pressure in the second chamber. For example, the pressure in the first chamber is one or two orders of magnitude lower than the pressure in the second chamber. Because of this pressure difference, any impurity atoms or molecules possibly sputtered by the high-energy ion beam can be sucked away from the surface back to the first chamber through the exit aperture and are pumped out. This inherently reduces the possibility of a re-deposition of the sputtered atoms and molecules back onto the implanting surface. Otherwise, the re-deposition of these atoms/molecules may alter the surface morphology and/or implantation profile. For example, some contaminate atoms on the surface may change the EOR of the implanted ion so that the defect network becomes scattered. In another example, the surface tends to become rough quickly and unable to be performed a repeated Direct Film Transfer process so to greatly lower the manufacture yield. In general the embodiments for keeping the pressure difference between the first and second chambers provides a method of low contaminate processing. Of course, there can be other variations, modifications, and alternatives.

According to an preferred embodiment, a system of introducing a linear accelerator particle beam for low contaminate processing includes a linear accelerator with at least an ion source for generating a plurality of charged particles and an apparatus for accelerating and confining the plurality of charged particles in a first particle beam in a first spatial direction. The first particle beam includes at least a first ion specie in an energy level ranging from 1 MeV to 5 MeV and is outputted to a first exit aperture. Additionally, the system includes a beam filter apparatus configured to receive the first particle beam. The beam filter apparatus includes a first chamber housing a mass-selection device to process the first particle beam and generate a second particle beam. The second particle beam includes substantially the first ionic specie only and being outputted to a second exit aperture in a second spatial direction that is different from the first spatial direction. In one embodiment, the first ionic specie is H⁺ ion. In another embodiment, the first ion specie is H₂⁺ ion. Moreover, the system includes an end-station with a second chamber configured to receive the second particle beam. The second chamber houses a pre-loaded workpiece with a planar surface configured to be irradiated by the second particle beam for implanting the first ionic specie. The first chamber is associated with a first pressure and the second chamber is associated with a second pressure that is higher than the first pressure. For example, the system is one of system 200, 200B, 300, or 300B.

According to an alternative embodiment, a system of introducing a linear accelerator particle beam for low contaminate processing includes a linear accelerator apparatus configured to generate a first particle beam comprising at least a first ionic specie in an energy level of at least 1 MeV. The system further includes a beam filter coupled to the linear accelerator apparatus to receive the first particle beam. The beam filter is in a first chamber and is configured to generate a second particle beam with substantially the first ionic specie only. In one embodiment, the first ionic specie is H⁺ ion. In another embodiment, the first ion specie is H₂⁺ ion. Moreover, the system includes an end-station with a second chamber coupled to the first chamber for extracting the second particle beam. The second particle beam is irradiated onto a planar surface of a workpiece loaded in the second chamber for implanting the first ionic specie. The first chamber is associated with a first pressure and the second chamber is associated with a second pressure that is higher than the first pressure. In one embodiment, the system is system 200 in FIG. 2A. in another embodiment, the system is system 300 in FIG. 3A.

According to still another alternative embodiment, the invention provides a method of introducing linear accelerator particle beam for low contaminate processing. The method includes generating a plurality of ionic particles by an ion source. The plurality of ionic particles contain multiple species including a first ion specie. Additionally, the method includes accelerating and confining the plurality of ionic particles to a first particle beam with energy level of the first specie at least in a range of 1 MeV to 5 MeV and higher using a linear accelerator. The method further includes extracting the first particle beam through a first exit aperture to a first chamber. The first chamber is associated with a first pressure. Moreover, the method includes processing the first particle beam in the first chamber to separate the first ion specie out of the multiple species and extracting a second particle beam through a second exit aperture into a second chamber. The second particle beam includes substantially the first ion specie only. In one embodiment, the first ionic specie is H⁺ ion. In another embodiment, the first ion specie is H₂⁺ ion. Furthermore, the method includes irradiating the second particle beam to one or more planar surfaces of one or more bulk workpieces loaded in the second chamber to implant the first ion specie. The second chamber is associated with a second pressure that is higher than the first pressure. In one preferred embodiment, the method is method 400 in FIG. 4.

Numerous benefits are achieved using embodiments of the present invention. In particular, embodiments of the present invention use a linear accelerator based on RFQ-linac and/or RFI technology that has been proven to be a cost effective way to obtain high-energy proton beam in at least 1 MeV. According to certain embodiments of the invention with proper dosage and temperature controls these high-energy H⁺ ions can be utilized for deep implantation down to 200 μm beneath a surface of a selected bulk semiconductor with minimum surface damage to form a desired cleave region thereof. Subsequently, through various controlled cleaving processes or direct layer transfer processes a free-standing thick film (with thickness about 200 μm or less) can be produced. Some embodiments of the invention can be used to produce free-standing single crystalline silicon or polycrystalline silicon thick films for manufacture photovoltaic cells.

Some other embodiments of the present invention provide a method of introducing high energy particles for ion implantation with a less contaminate process. The method utilizes a beam filter to separate a desired ionic specie, for example, the H⁺ ion, from other ionic species with different mass or charge, so that the less contaminate ions will be implanted which otherwise may cause wide-spreading defects instead of forming the cleave region as a predominant 2-D defect network. Additionally, the beam filter according to certain embodiments of the invention can bend the beam angle, either horizontally or vertically, providing a geometric flexibility for system arrangement. Particularly, the end-station is such a system can be easily incorporated into a cluster processing tool. Some specific embodiments of the present invention also provide method of performing ion implantation with a less contaminate process by setting a pressure difference between the end-station chamber and the beam filter chamber so that any impurity atoms or molecules sputtered by the particle beam can be prevented from re-depositing onto the implanting surface. Depending upon the embodiment, one or more of these benefits may be achieved.

At least the following embodiments are understood as following within the scope of the present invention.

A system wherein the first chamber is associated with a first pressure and the second chamber is associated with a second pressure, the first pressure being set to be at least 10× lower relative to the second pressure. A system wherein the second pressure in the second chamber is about (1×10⁻³) torr and lower.

A system wherein ion source is an electron cyclotron resonance (ECR) ion source. A system wherein the apparatus for accelerating and confining the plurality of charged particles in a first particle beam is a linear accelerator (linac) system. A system wherein the linac system comprises a stage selected from a radio frequency quadrupole (RFQ) stage, a RF-Focused Interdigitated (RFI) stage, a Drift-Tube Linac (DTL) stage, or a Quadrupole-Focused Interditigated (QFI) stage. A system wherein the apparatus for accelerating and confining the plurality of charged particles in a first particle beam comprises multiple stages.

A system wherein the first particle beam is in an energy level at least 1 MeV. A system wherein the first particle beam is in an energy level ranging from 0.5 MeV to 5 MeV.

A system wherein the first particle beam comprises the first ionic specie and a plurality of contaminate species originated from either the ion source or generated during the propagation through the linear accelerator. A system wherein the first ion specie is H+ ion. A system wherein the first ion specie is H2+ ion. A system wherein the plurality of contaminate species is selected from the group of Hydrogen isotope specie, Helium specie, Oxygen specie, Nitrogen specie, Carbon specie, Aluminum Specie, Iron Specie, Copper Specie, and constituent elements of aluminum and steel alloys.

A system wherein the mass-selection device comprises an electric sector. A system wherein the mass-selection device comprises a magnetic sector. A system of claim 1 wherein the mass-selection device comprises any combination of an electric sector, a magnetic sector, electric/magnetic (E×B), and one or more drift channels. A system wherein the mass-selection device is based on a mechanism of generating separate trajectories with different radius of curvature for particles with different charge-to-mass ratio. A system wherein the mass-selection device is based on a mechanism for differentiating particles by charge-to-mass ratio.

A system wherein the first spatial direction relative to the second spatial direction forms an angle in a horizontal plane. A system wherein the angle is about 90 degrees. A system wherein the angle is about 45 degrees. A system wherein the angle is about 135 degrees. A system wherein the first spatial direction relative to the second spatial direction forms an angle in a vertical plane. A system wherein the angle is about 90 degrees.

A system wherein the beam filter apparatus is a two-stage mass-selection device further comprising a third chamber housing a second mass-selection device coupled to the first mass-selection device in series to produce the second particle beam. A system wherein the third chamber is associated with a third pressure that is set to be lower relative to the second pressure.

A system wherein the end-station including a second chamber coupled to the second exit aperture is configured to receive the second particle beam in horizontal direction. A system wherein the end-station is part of a cluster tool configured to integrate with a plurality of chambers including the second chamber. A system wherein the second chamber is configured to couple with one or more load locks for loading in/out the workpiece.

A system wherein the workpiece is a thick film on a substrate, a tile workpiece, a wafer workpiece, or a bulk workpiece. A system wherein the bulk workpiece comprises a shaped ingot of single-crystalline or polycrystalline silicon, germanium, III/V group compound semiconductor.

A system wherein the second chamber is further configured to load/unload a plurality of bulk workpieces each including a planar surface for batch processing. A system wherein the second chamber is configured to allow each planar surface of the plurality of bulk workpieces being irradiated by the extracted second particle beam in a substantially perpendicular direction. A system wherein the plurality of bulk workpieces are loaded on a rack that is movable in a plane allowing the second particle beam effectively scanning over all the planar surfaces of the plurality of bulk workpieces.

A system wherein the end-station including a second chamber coupled to the first chamber is configured to receive the second particle beam in horizontal direction. A system wherein the end-station including a second chamber coupled to the first chamber is configured to receive the second particle beam in vertical direction.

As described in detail above, various embodiments of the present invention relate to methods and apparatuses for processing a workpiece with a particle beam. One potentially important application for such processing is in the performance of a controlled cleaving process. For example, in one embodiment, hydrogen, helium, and/or a rare gas are implanted into a substrate to define a subsurface cleave region, followed by a controlled cleaving action to separate a thin film of material from the implanted substrate. Such a controlled cleaving process is described more fully in U.S. Pat. No. 6,013,563 incorporated by reference in its entirety herein for all purposes. A system for achieving such controlled cleaving may include one or more energy sources configured to achieve initiation and propagation of a cleave front in the substrate. Examples of such energy sources include but are not limited to a thermal source or sink, a chemical source, a mechanical source, and an electrical source.

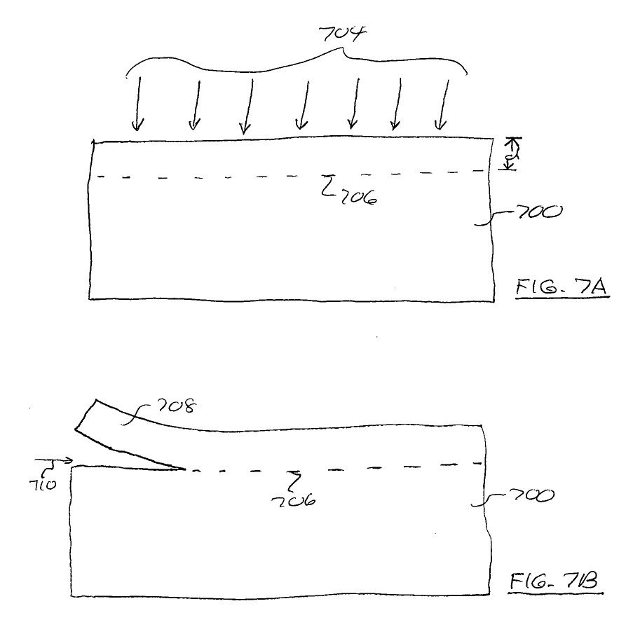

FIGS. 7A-B show simplified cross-sectional views of a controlled cleaving process according to an embodiment of the present invention. Specifically, in FIG. 7A, a surface 702 of a substrate or workpiece 700 is exposed to particles from a particle beam 704 that are implanted to form subsurface cleave region 706 at a depth d into the substrate. FIG. 7B shows the initiation and propagation of a controlled cleaving action to separate a film 708 from the substrate. Application of energy 710 may be useful in the initiation and/or propagation of the controlled cleaving process.

It is understood that the examples and embodiments described herein are for illustrative purposes only and that various modifications or changes in light thereof will be suggested to persons skilled in the art and are to be included within the spirit and purview of this application and scope of the appended claims.

What is claimed is:

1. A system of introducing a linear accelerator particle beam for low contaminate processing, the system comprising:
a particle accelerator including at least an ion source for generating a plurality of charged particles, and an apparatus for accelerating and confining the plurality of charged particles in a first particle beam, the first particle beam being outputted to a first exit aperture in a first spatial direction;
a beam filter apparatus coupled to the first exit aperture to receive the first particle beam, the beam filter apparatus including a first chamber of a first mass-selection device to process the first particle beam and generate a second particle beam, the second particle beam including substantially a first ionic specie and being outputted to a second exit aperture in a second spatial direction different from the first spatial direction;
an end-station including a second chamber coupled to the beam filter apparatus to receive the second particle beam, the second chamber configured to house a workpiece having a planar surface configured to receive the second particle beam for implanting the first ionic specie.

2. The system of claim 1 further comprising an apparatus configured to perform a controlled cleaving of a film of material from the workpiece, the film having a thickness corresponding to a depth of implantation of the particles from the second beam.

3. The system of claim 1 wherein the first chamber is associated with a first pressure and the second chamber is associated with a second pressure, the first pressure being set to be at least 10× lower relative to the second pressure.

4. The system of claim 3 wherein the second pressure in the second chamber is about (1×10⁻³) torr and lower.

5. The system of claim 1 wherein the apparatus for accelerating and confining the plurality of charged particles in a first particle beam is a linear accelerator (linac) system.

6. The system of claim 5 wherein the linac system comprises a stage selected from a radio frequency quadrupole (RFQ) stage, a RF-Focused Interdigitated (RFI) stage, a Drift-Tube Linac (DTL) stage, or a Quadrupole-Focused Interditigated (QFI) stage.

7. The system of claim 1 wherein the apparatus for accelerating and confining the plurality of charged particles in a first particle beam comprises a cyclotron or a DC electrostatic particle accelerator.

8. The system of claim 1 wherein the first particle beam is in an energy level ranging from 0.5 MeV to 5 MeV.

9. The system of claim 1 wherein the first particle beam comprises the first ionic specie and a plurality of contaminate species originated from either the ion source or generated during the propagation through the linear accelerator.

10. The system of claim 9 wherein the plurality of contaminate species is selected from the group of Hydrogen isotope specie, Helium specie, Oxygen specie, Nitrogen specie, Carbon specie, Aluminum Specie, Iron Specie, Copper Specie, and constituent elements of aluminum and steel alloys.

11. The system of claim 1 wherein the mass-selection device comprises a magnetic sector, an electric sector, or any combination of an electric sector, a magnetic sector, electric/magnetic (E×B), and one or more drift channels.

12. The system of claim 1 wherein the mass-selection device is based on a mechanism for differentiating particles by charge-to-mass ratio.

13. The system of claim 1 wherein the first spatial direction relative to the second spatial direction forms an angle in a horizontal plane or in a vertical plane.

14. The system of claim 13 wherein the angle is about 45 degrees, about 90 degrees, or about 135 degrees.

15. The system of claim 1 wherein the beam filter apparatus is a two-stage mass-selection device further comprising a third chamber housing a second mass-selection device coupled to the first mass-selection device in series to produce the second particle beam.

16. The system of claim 15 wherein the third chamber is associated with a third pressure that is set to be lower relative to the second pressure.

17. The system of claim 1 wherein the end-station is part of a cluster tool configured to integrate with a plurality of chambers including the second chamber.

18. The system of claim 17 wherein the second chamber is configured to couple with one or more load locks for loading in/out the workpiece.

19. The system of claim 1 wherein the workpiece is a thick film on a substrate, a tile workpiece, a wafer workpiece, or a bulk workpiece.

20. The system of claim 19 wherein the bulk workpiece comprises a shaped ingot of single-crystalline or polycrystalline silicon, germanium, III/V group compound semiconductor.

21. The system of claim 20 wherein the plurality of bulk workpieces are loaded on a rack that is movable in a plane allowing the second particle beam effectively scanning over all the planar surfaces of the plurality of bulk workpieces.

22. A system of introducing an accelerator particle beam for low contaminate processing, the system comprising:
a particle accelerator apparatus configured to generate a first particle beam;
a beam filter coupled to the linear accelerator apparatus to receive the first particle beam, the beam filter being in a first chamber and configured to generate a second particle beam with substantially a first ionic specie;
an end-station including a second chamber coupled to the first chamber for extracting the second particle beam, the second particle beam being irradiated onto a planar surface of a workpiece loaded in the second chamber for implanting the first ionic specie.

23. The system of claim 1 further comprising an apparatus configured to perform a controlled cleaving of a film of material from the workpiece, the film having a thickness corresponding to a depth of implantation of the particles from the second beam.

24. The system of claim 22 wherein the first chamber is associated with a first pressure and the second chamber is associated with a second pressure, the first pressure lower relative to the second pressure.

25. The system of claim 24 wherein the second pressure is about 1×10⁻³ torr and lower.

26. The system of claim 22 wherein the particle accelerator comprises a RF accelerator such as an RF linac or a cyclotron, or comprises a DC electrostatic accelerator.

27. The system of claim 22 wherein the first particle beam is in an energy level ranging from 1 MeV to 5 MeV.

28. The system of claim 22 wherein the first particle beam comprises the first ionic specie and a plurality of contaminate species originated from either the ion source or generated during the propagation through the linear accelerator.

29. The system of claim 22 wherein the plurality of contaminate species is selected from Hydrogen isotope specie, Helium specie, Oxygen specie, Nitrogen specie, Carbon specie, Aluminum specie, Iron specie, Copper specie, elements of aluminum and steel alloys.

30. The system of claim 22 wherein the beam filter comprises a magnetic sector, an electric sector, or any combination of an electric sector, a magnetic sector, electric/magnetic sectors, and one or more drift channels for separating the first ionic specie from the first particle beam.

31. The system of claim 22 wherein the beam filter is based on a mechanism of generating separate trajectories with different radius of curvature for particles with different charge-to-mass ratio.

32. A method of introducing a accelerator particle beam for low contaminate processing, the method comprising:
generating a plurality of ionic particles by an ion source, the plurality of ionic particles comprising multiple species including a first ionic specie;
accelerating and confining the plurality of ionic particles to a first particle beam with energy level of the first specie at least in a range of 0.5 MeV to 5 MeV using a linear accelerator;
extracting the first particle beam into a first chamber;
processing the first particle beam in the first chamber to separate the first ion specie from the multiple species;
extracting a second particle beam into a second chamber, the second particle beam comprising substantially the first ion specie only;
irradiating the second particle beam to implant the first ion specie into one or more planar surfaces of one or more bulk workpieces loaded in the second chamber.

33. The method of claim 32 wherein the accelerating and confining the plurality of ionic particles to a first particle beam comprises:
extracting the plurality of ionic particles from the ion source through a low energy beam transport unit;
accelerating and confining the plurality of ionic particles in a multi-stage radio frequency (RF) quadrupole (RFQ) linear accelerator (linac), a cyclotron, or a DC electrostatic accelerator; and
generating the first particle beam.

34. The method of claim 32 wherein the processing the first particle beam in the first chamber to separate the first ion specie from the multiple species further comprises:
receiving the first particle beam;
guiding the first particle beam into a beam filter;
guiding the first ionic specie through the beam filter while dumping the rest of the multiple species.

35. The method of claim 34 wherein the beam filter comprises a mass-selection device based on a mechanism for differentiating particles by charge-to-mass ratio.

36. The method of claim 35 wherein the charge-to-mass selection device comprises a combination of one or more electric sectors, one or more magnetic sectors, and one or more drift channels.

37. The method of claim 32 wherein the extracting a second particle beam into a second chamber comprises:
receiving a plurality of first ionic specie particles to form a second particle beam;
outputting the second particle beam through a high-energy beam transport unit at a second exit aperture of the first chamber, the second exit aperture being connected to the second chamber;
expanding optionally the second particle beam to obtain a desired beam diameter.

38. The method of claim 32 wherein the irradiating the second particle beam to implant the first ion specie into one or more planar surfaces comprises:
directing the second particle beam to a spot of the one or more planar surfaces in substantially perpendicular direction;
scanning the second particle beam to move the spot over the entire one or more planar surfaces;
controlling a dosage by adjusting at least a beam current, a beam diameter, and a scanning speed.

39. The method of claim 32 wherein the one or more bulk workpieces can be ingots of single-crystalline or polycrystalline silicon, germanium, III/V group compound semiconductor, or silicon carbide (SiC).

40. The method of claim 32 wherein the second chamber belongs to a cluster tool configured to perform other processes including surface re-polishing, post-processing, and controlled cleaving or direct film transferring.

41. The method of claim 32 wherein the first chamber is associated with a first pressure and a second chamber is associated with a second pressure.

42. The method of claim 41 wherein the second pressure is about 1×10⁻³ torr or lower.

43. The method of claim 42 wherein the first pressure is one or two orders of magnitude lower than the second pressure.

44. The system of claim 1 further comprising a beam scanner configured to alter over time a location that the second particle beam impinges upon the workpiece.

45. The method of claim 32 further comprising translating the bulk workpieces along at least one axis during impingement of the second particle beam.

46. An apparatus comprising:
a linear accelerator having an inlet in vacuum communication with an ion source and an outlet in vacuum communication with an inlet of a beam filter;
an end station in vacuum communication with an outlet of the beam filter and configured to support a target workpiece;
a host computer comprising,
a processor in electronic communication with at least one element selected from the ion source, the linear accelerator, the beam filter, and the end station, and
a computer readable storage medium in electronic communication with the processor and having stored thereon code configured to instruct the processor to,
cause the ion source to generate a plurality of ionic particles comprising multiple species including a first ionic specie,
cause the linear accelerator to accelerate the plurality of ionic particles to a particle beam with energy level of the first specie at least in a range of 0.5 MeV to 5 MeV,
cause the beam filter to process the particle beam in the first chamber to separate the first ion specie from the multiple species, and
irradiate the particle beam to implant the first ion specie into one or more planar surfaces of one or more workpieces loaded in the end station.

47. The apparatus of claim 46 wherein the computer readable storage medium further comprises code to instruct scanning of the first ion species over the surface of the workpiece during the irradiation.

48. The apparatus of claim 46 wherein the computer readable storage medium further comprises code to instruct translation of the workpiece along one or more axes during the irradiation.

49. The apparatus of claim 46 wherein:
the beam filter comprises a first chamber magnetic communication with an analyzing magnet; and
the computer readable storage medium further comprises code to instruct the analyzing magnet to apply a magnetic field to the first chamber.

50. The apparatus of claim 46 wherein the computer readable storage medium further comprises code to instruct that the first chamber to be maintained at a first pressure lower than a second pressure of the end station.

51. A method of forming a thin film, the method comprising:
generating a high energy particle beam;
passing the high energy particle beam through a filter to remove unwanted contamination from the particle beam;
directing the decontaminated particle beam at a surface of a substrate;
forming a cleave region in the substrate from particles implanted from the beam; and
performing a controlled cleaving in the cleave region to remove a thin film of material from the substrate.

52. The method of claim 51 wherein a thickness of the thin film is at least about 10 μm.
